(19) United States
(12) Patent Application Publication
(10) Pub. No.: US 2009/0221916 A1
(43) Pub. Date: Sep. 3, 2009

(54) Systems and Methods for Elastography Imaging

(75) Inventors: Elisa E. Konofagou, New York, NY (US); Helen Lu, New York, NY (US); Simon Fung-Kee-Fung, Buffalo, NY (US); Daniel Ginat, New York, NY (US); Jeff Spalazzi, Fair Lawn, NY (US)

Correspondence Address: KRAMER LEVIN NAFTALIS & FRANKEL LLP;INTELLECTUAL PROPERTY DEPARTMENT, 1177 AVENUE OF THE AMERICAS, NEW YORK, NY, 10036, US

(73) Assignee: The Trustees of Columbia University in the City of New York, New York, NY (US)

(21) Appl. No.: 12/096,254

(22) PCT Filed: Dec. 8, 2006
(86) PCT No.: PCT/US2006/061809

§ 371 Date: Nov. 26, 2008

Related U.S. Application Data
(60) Provisional application No. 60/749,245, filed on Dec. 9, 2005.
(60) Provisional application No. 60/751,122, filed on Dec. 16, 2005.

(51) Int. Cl. A61B 8/14

(52) U.S. Cl. 600/443

(57) ABSTRACT

Methods for obtaining information about the mechanical behaviour of structures associated with mammalian joints and tendons are provided. Embodiments of such methods include creating deformation in a joint structure (such as ligaments and articular cartilage) or tendon of interest, using an ultrasound scanner and a single element or array of elements to acquire sequences of ultrasound data of the joint structure or tendon, estimating one, two or three components of the resulting displacement and strain between a reference frame of ultrasound data and successive frames of ultrasound data, and using a cross-correlation algorithm to estimate the displacement and strain components. This information may be used to inform the design of tissue grafts. Tissue grafts produced using this information are also provided. The same method can be used in situ together with noninvasive or invasive procedures.

### RELATED APPLICATIONS

This application claims priority to and benefit of U.S. provisional application No. 60/749,245 filed Dec. 9, 2005 and U.S. provisional application No. 60/751,122 filed Dec. 16, 2005, the contents of each of which is incorporated herein in its entirety.

### FIELD OF THE INVENTION

The present invention relates to the use of elastography in the mechanical characterization of anatomical structures associated with mammalian joints and tendons.

### BACKGROUND

Elasticity imaging, or methods for the mapping of mechanical responses or properties using ultrasound or MRI images acquired before and after a mechanical excitation, was initially developed in the early 1990s as an alternative tool for early tumor diagnosis based on the principle of palpation. It has since been utilized in intravascular and cardiovascular applications in vivo, as well as in the guidance of thermal therapy procedures and robotic surgery. Elastography has also been used to determine mechanical properties of tissues by measuring both elastic and viscoelastic properties.

Despite these recent advances, a need still exists for obtaining detailed mechanical characterization of joint structures and tendons in both human and veterinary medicine. For example, detailed characterization of the manner in which a human anterior cruciate ligament (hereinafter “ACL”) responds to strain would be beneficial in designing appropriate replacement grafts. In the United States alone approximately 75,000-100,000 ACL reconstructions are performed each year.

Due to the poor healing potential of the ACL, surgical intervention is often required following ligament injury. Surgery most often constitutes complete reconstruction of the ACL using an autologous tendon graft. Two autografts commonly utilized to replace the ACL are the bone-patellar tendon-bone (BPTB) graft and the semitendinosus or hamstring tendon (HT) graft. While the BPTB graft, which consists of the central third of the patellar tendon, has the advantage of possessing bony ends that facilitate integration with bone in the femoral and tibial bone tunnels, harvesting of these autografts often results in significant donor site morbidity. Consequently, there has been a shift toward the usage of HT grafts, which are the most commonly used grafts for ACL reconstruction. While harvesting of HT grafts results in significantly less donor site morbidity, these grafts are mechanically anchored, and their clinical success is limited by the lack of biological graft integration with the subchondral bone.

Native ACL inserts into bone through a direct insertion consisting of a linear transition from ligament to fibrocartilage to bone. The fibrocartilage zone is further divided into non-mineralized and mineralized fibrocartilage regions. Due to the presence of several types of tissue, the ACL-bone interface is expected to vary in cellular, chemical, and mechanical properties. It is believed this controlled heterogeneity permits the transition of mechanical load between bone and soft tissue and minimizes the formation of stress concentrations. This interface, however, is not re-established after tendon graft-based ACL reconstruction. Without a stable interface, the fixation site of the HT grafts to bone becomes the weak link in the reconstructed graft, a leading cause of graft failure and resulting in revision surgery.

Promoting clinically successful graft-bone integration via the regeneration of the native ligament-bone interface of the ACL (or other joint structures) requires a detailed understanding of the mechanical properties of both the ligament and the ligament-bone interface. This would allow for the proper consideration of material selection and interface regeneration via tissue engineering methods. Elucidating the structure-function relationship at the insertions would be critical in the design of the next generation of graft fixation devices, enabling biological fixation of tendon grafts to bone through the reestablishment of the native tendon to bone interface.

Osteoarthritis is another condition that affects many millions of people worldwide. Osteoarthritis is a disease process involving articular cartilage. Articular cartilage is poroelastic and bears load in articular joints. The use of radiography and physical examination to examine nascent osteoarthritis is quite limited, however.

Consequently, there have been attempts to develop arthroscopic indentation devices that enable measurement of the biomechanical properties of degenerate cartilage, with the aim of early diagnosis. In particular, some efforts have been made to combine arthroscopic indenters with ultrasound probes, mainly to obtain more accurate estimates of cartilage thickness. But in order for arthroscopy to become the gold standard for diagnosing cartilage pathology, data acquisition and presentation must be improved upon greatly.

Detailed mechanical characterization, data acquisition and presentation of structures associated with joints (such as ligaments, cartilage and menisci) and tendons would be very beneficial.

### SUMMARY

The present invention includes the use of ultrasound elastography to determine strain distribution of joint structures and tendons. Joints may include but are not limited to those of the foot, ankle, hip, temporomandibular joint (TMJ), shoulder, elbow, hand and wrist and corresponding anatomical structures in non-human mammals. Joint structures may include, for example, ligaments such as the ACL, cartilage (especially articular cartilage), and the medial and lateral menisci of, for example, the tibiofemoral joint. Tendons may include, for example, the achilles tendon and flexor and extensor tendons of mammalian extremities.

Exemplary embodiments of the present invention provide methods for obtaining information about the mechanical behaviour of structures associated with mammalian joints where such methods include, creating deformation in a joint structure of interest, using an ultrasound scanner and a linear array to acquire sequences of ultrasound data of the joint structure, and estimating the axial displacement between a reference frame of the data and successive frames of the data. Deformation may be either active or passive and can include, for example, tension, compression, relaxation and combination thereof.

Another exemplary embodiment of the present invention provides methods for obtaining information about the mechanical behaviour of tendons wherein such methods include, creating deformation in a tendon of interest, using an ultrasound scanner and a linear array to acquire sequences of ultrasound data of the tendon, and estimating the axial displacement between a reference frame of the data and successive frames of the data. Again, deformation may be either active or passive and can include, for example, tension, compression, relaxation and combination thereof.

In yet another exemplary embodiment, the present invention provides methods which include estimating axial displacement and strain using a ID cross-correlation algorithm.

In still another exemplary embodiment, the present invention provides methods which include estimating 2D and/or 3D axial displacement and strain.

In another exemplary embodiment, the present invention includes the use of cross-correlation algorithms to determine time-shifts between two backscattered signals by cross-correlating sliding windows over a 2D ultrasound image.

A further exemplary embodiment of the present invention includes using information concerning the mechanical characterization of structures associated with joints and tendons to inform the design of tissue grafts.

Exemplary embodiments of the present invention also include tissue grafts produced using information obtained by the mechanical characterization of structures associated with joints and tendons.

Exemplary embodiments of the present invention also allow for imaging of displacement and strain as well as estimation of displacement and strain.

### BRIEF DESCRIPTION OF THE DRAWINGS

FIG. 1 illustrates a flow chart for a representative recorrelation technique.

FIGS. 2A and 2B provide a representative image produced by tracking an RF segment in 2D for axial (Δs) and lateral (Δ1) displacement estimation. A's and B's are RF lines corresponding to consecutive frames in time.

FIG. 3A shows an exemplary equipment configuration for ultrasound data acquisition during tensile testing. Neonatal bovine patellofemoral joint is loaded on a mechanical testing system (MTS) modified with a cylindrical polycarbonate tank. The tank is filled with physiologic saline and the ultrasound transducer (arrow) is mounted inside the tank such that the ACL and insertions can be scanned posteriorly.

FIG. 3B shows the neonatal bovine patellofemoral joint shown in FIG. 3A mounted in the MTS with a tibial orientation and 0° flexion.

FIG. 4A shows a posterior view of scanned ACL and insertions.

FIG. 4B shows an ultrasound image of tibial insertion.

FIG. 4C shows a corresponding y-displacement map (mm), with blue to red indicating small to large displacements, respectively.

FIG. 4D shows a corresponding elastogram with compressive strain (not in %) indicated by blue regions and tensile strain by red regions. Negative values denote compressive strain.

FIGS. 5A and B show, respectively, displacement (5A) and strain (5B) at the tibial insertion as collected from point-wise temporal analysis. Point of data analysis is indicated by the arrow shown in FIG. 4.

FIG. 6 shows sequences (1-16) of a) displacement images and b) elastograms at 15 frames apart during tensile loading. Blue denotes high compressive strain and red denotes high tensile strain on the elastograms. Development of high displacements in the ligament compared to bone during tensile loading are shown as indicated by the change from blue to rod in the ligament proper.

FIG. 7A shows an ultrasound image of an ACL and insertions with the transducer rotated so that the face of the transducer was aligned along the principal axis of the ACL.

FIGS. 7B and 7C show, respectively, axial displacement (mm) (7B) and strain (not in %) (7C) during tensile stretching in the lateral direction versus the axial beam propagation. A compressive (blue) strain can be seen in 7B corresponding to the ACL-bone interface (orange region in FIG. 7A).

FIG. 8 shows a representative design of a compression apparatus and image acquisition arrangement for elastographic imaging of cartilage.

FIG. 9 graphically illustrates a representative load versus time for an imposed pre-strain of 10% followed by an additional 2% strain 90 seconds later demonstrating stress-relaxation of articular cartilage. The load axis is on the right of the graph and the strain axis is on the right.

FIGS. 10A and 10B show, respectively, a representative grey-scale RF signal of a cartilage sample within a compression apparatus with a ⅛″ inch opening (10A) and a 1/32″ opening (10B). In FIGS. 10A and 10B the bracket represents the opening in the loading plate, while the arrow show the highly reflective metal loading plate.

FIG. 11A provides a representative displacement image of a femoral condyle cartilage sample showing uniform displacement in the region of interest, using a ⅛″ opening with a scale ranging from −0.1 mm to 0.1 mm.

FIG. 11B provides a representative elastogram of the same sample shown in FIG. 11A where the sample shows essentially zero local strain except at the surface and interface of zone 1 and 2 with a scale ranging from −0.9% to 0.9%.

FIG. 12A shows a representative displacement image of a femoral condyle cartilage sample showing a slight displacement gradient in the region of interest, using a 1/32″ opening. The loading plate is indicated by an arrow. The scale ranges from −0.1 mm to 0.1 mm.

FIG. 12B shows a representative elastogram of the same sample shown in FIG. 12A where the sample shows essentially zero local strain except at the surface and interface of zone 1 and 2 with a scale ranging from −0.2% to 0.2%.

### DETAILED DESCRIPTION OF EXEMPLARY EMBODIMENTS

Understanding the mechanical behavior exhibited by joint structures and tendons is crucial in the diagnostic evaluation of conditions created by pathology and/or injury In addition, such understanding is necessary for the logical implementation of replacement grafts as well as in the design of biomimetic scaffold systems.

For example, understanding the mechanical behavior of native ACL and insertions provides valuable information concerning the native functional ligament interface at the junctions between the graft and bone. Currently, the long term integrity of semitendinosus and hamstring tendon grafts used for ACL reconstruction is limited by healing of the graft with bone, which results in non-anatomical fibrovascular scar tissue at the interface between the tendon graft and bone. The success of ACL grafts depends on reforming the native anatomical tendon-bone interface. Methods according to the present invention allow for the determination of the structure-function relationship of joint structures, such as the ACL. This information then may be used to design scaffold systems, such as tendon grafts to bone, which mimic the native tissue in morphology, chemical composition, cellular distribution, and mechanical properties.

Methods according to the present invention are particularly useful for characterizing strain applied to structures associated with joints such as ligaments and cartilage, as well as tendons. Much of the description below and in the Examples section is directed to methods involving characterization of the anterior cruciate ligament and the characterization of articular cartilage of the femoral condyles. It should be understood, however, that these methods are readily applicable to other anatomical structures, for example, other ligaments, cartilage and tendons, for which detailed characterization of strain responses is desired.

These methods are especially appropriate where such structures are subject to non-uniform strain and may be used in vitro, in situ and under minimally invasive conditions. Minimally invasive conditions include, for example, endoscopic procedures. Methods according to the present invention also can be used for veterinary applications in, for example, equine, canine and feline species.

Methods according to the present invention are ideal for examining, for example, the ACL-bone interface as such methods permit the characterization of relatively small areas (on the order of about 0.1-2 mm, depending on the ultrasound frequency used) with complex stress distributions. In an exemplary embodiment, an ultrasound transducer scans a region of interest while an external load is applied to induce strain. Speckle tracking techniques may be employed to analyze the collected radio-frequency ultrasonic data before and after incremental loading and to estimate the resulting strain and strain distributions. Standard ultrasound scanners (e.g. Terason 2000, Teratech, Framingham Mass.) or similar devices may be used. Ultrasound frequencies may range from, for example, 2-40 MHz, although higher or lower frequencies may be appropriate in certain circumstances, as will be appreciated by those of ordinary skill in the art. Sequences of RF data may be acquired during loading of the joint. Axial displacement between a reference and successive frames can be estimated using cross-correlation and recorrelation techniques, exemplary embodiments of which are described in more detail below.

Axial displacements, or displacements occurring in the direction orthogonal to the face of the transducer and parallel to the direction of ultrasound propagation can be estimated for each RF frame with respect to a reference frame by using a 1D cross-correlation algorithm. Strain distribution can be computed by differentiating the displacement map along the axial direction. For numerical differentiation, a least-squares regression method may be used. Displacement and strain then may be estimated relative to a reference frame to obtain a temporal profile and map of the cumulative deformation at the ligament and the insertion. These methods may be used to generate maps of cumulative deformation and strain in, for example, the ACL and tibial insertion during tensile loading. These techniques can be used to generate detailed information about displacements and strains associated with the tibiofemoral joint and other joints and tendons.

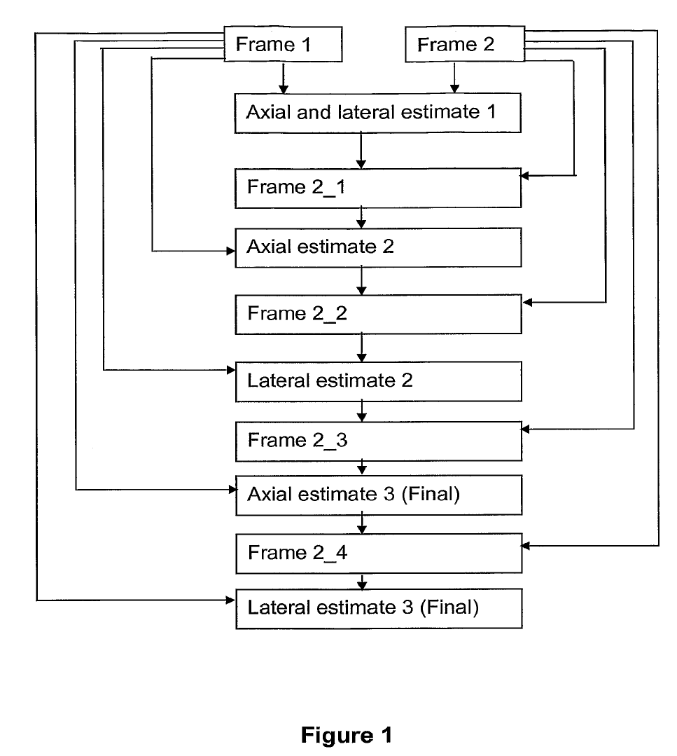

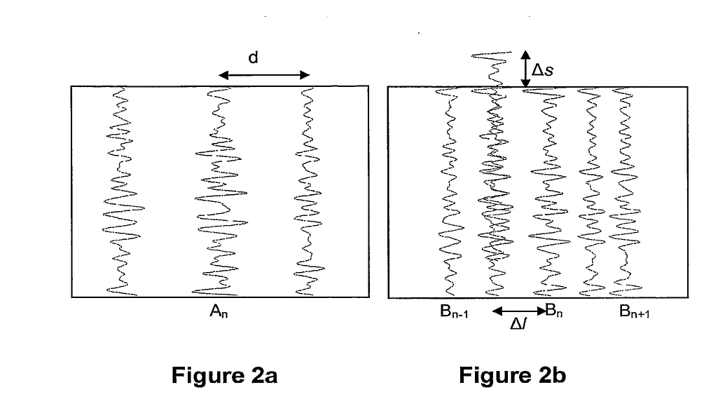

Cross-correlation and recorrelation algorithms may be used to obtain detailed information as described below. FIG. 1 provides a representative sequence of cross-correlation and recorrelation algorithms. These techniques may be applied between consecutive echo (RF, envelope-detected or B-mode) axial (1) segments in 2D (in-plane) or 3D (in-plane and out-of-plane). In a representative embodiment, each frame may be considered as a set of echo segments. As shown in FIGS. 2A and 2B each echo segment in a first frame may be cross-correlated with echo segments in a second frame to find the best match. After the best match is found between axial segments Aₙ (FIG. 2A) and Bₙ (FIG. 2B) the 2D or 3D resultant that denotes the path of motion between segments 1 and 2 may be broken into its individual components in the axial (in-plane, along the propagation axis; Δs in FIG. 2), lateral (in-plane, orthogonal to the propagation axis; Δ1 in FIG. 2) and elevational (out-of-plane). In its first iteration, the same technique is applied after recorrelation, i.e., after correction, or removal, for the motion occurring in a direction perpendicular to that of estimation. This provides an increase of the correlation coefficient and, thus, increases the signal-to-noise ratio associated with the resulting motion estimate. As a result, the largest source of decorrelation is removed, which is typically in the direction of deformation. For example, if the direction of mechanical tension is within the ultrasound image plane but in the lateral direction, the lateral component may be removed first from original Frame 2 (i.e., frame 2_1 may be generated) for the estimation of the new axial component (axial estimate 2; FIG. 1) and the second lateral component may be estimated (lateral estimate 2; FIG. 1) after removal of the new axial component (i.e., frame 2_2 may be generated) and so on until reaching high enough correlation for the iterations to no longer be useful (FIG. 1). This approach allows for effective decoupling of the 2D or 3D components and higher quality displacement and strain estimates and images of the tissue under deformation. It should be noted that the cross-correlation technique also may be employed without the use of recorrelation methods in the case where estimation of one or all components is considered to be of sufficient quality (e.g., high signal-to-noise ratio or high contrast-to-noise ratio).

The algorithms described immediately above can be modified in some circumstances for improved implementation of the invention, as will be appreciated by those of ordinary skill in the art. In addition, cross-correlation and recorrelation algorithms also may include those described in U.S. Pat. No. 6,270,459, the contents of which are incorporated by reference in their entirety.

It also should be noted that non-RF data such as B-mode and envelope-detected data may be obtained and applied to methods according to the present invention. 2D and 3D displacement and strain estimation also may be obtained by utilizing methods described herein.

Information generated by methods according to the present invention may be used to inform the design and material selection for the production of tissue grafts. Information on biodegradable scaffolds can be found in the literature, including Lu et al., Biomaterials 26 (2005) 4805-4816, the contents of which are incorporated herein by reference in its entirety.

Methods according to the present invention allow for graft designs and material selection to take into consideration the detailed strain response for a particular anatomical structure, such as an ACL. Both graft design and material selection are very important for long term clinical success and involve a balance between scaffold degradability, structural integrity needed for cell structure, overall scaffold mechanical properties, and the rate of cell matrix production.

Exemplary materials include those comprising poly-alpha-hydroxyesters such as polyglycolic acid (PGA), poly-L-lactic acid (PLLA), and polylactic-co-glycolic co-polymer (PLAGA), all of which have been approved by the FDA for these purposes. These types of degradable polymers do not elicit a permanent foreign body reaction and are gradually reabsorbed and replaced by natural tissue.

Protein modification of biomaterials may be used to improve cell adhesion and control the subsequent cellular response to material surfaces. Current strategies in improving cell attachment and augmenting subsequent cellular response include pre-coating these surfaces with molecules such as laminin, fibronectin (Fn) or grafting the Fn-related arginine-glycine-aspartic acid (RGD) tripeptide on biomaterials.

In addition to detailed characterization of ACL, the present invention also allows for high resolution ultrasound elastography of articular cartilage using the techniques described above and as discussed further in the Examples section below.

### EXAMPLES

### Example 1

### Materials and Methods Tissue Isolation and Joint Preparation

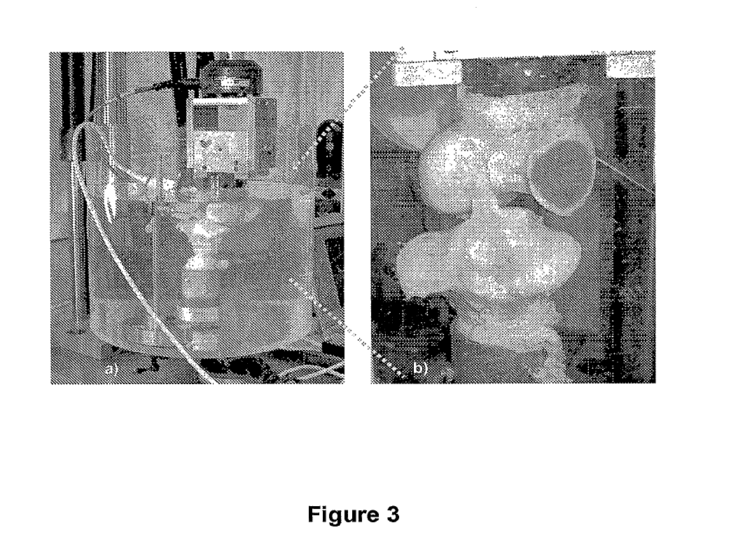

Neonatal bovine calf (up to one week old) tibiofemoral joints obtained from an abattoir (Fresh Farm Beef, Vermont) were used for this Example. After removal of surrounding muscle and adipose tissue, the joint capsule was opened. Fascia lata and connective tissue were removed from the joint capsule with the ACL and posterior cruciate ligament (PCL) undisturbed. The PCL was maintained intact until immediately prior to testing in order to maintain joint stability and prevent premature damage to the ACL. During all joint preparation procedures, the ACL and surrounding tissues were kept hydrated with physiologic saline. The femur and tibia were cut to approximately 12 cm from the joint with a hacksaw, the periosteum removed, and bone marrow extracted from the intramedullary cavity to improve cement fixation of the joint. Subsequently, the tibia and femur were secured with custom anchors and cement to prevent slippage during testing. The joint was then mounted on a uniaxial material testing system (MTS 858 Bionix Testing System; MTS, Eden Prairie, Minn.) fitted with a custom cylindrical polycarbonate tank, the PCL was severed, and the medial femoral condyle was removed with a hacksaw to improve line-of-sight access to the ACL and insertions for the ultrasound transducer (FIGS. 3A and 3B). These procedures may be applied in vivo for veterinary medicine purposes or in humans for assessing injury or age-related diseases such as osteoporosis.

### Materials and Methods Tensile Testing

Tensile testing was performed with the femur-ACL-tibia complex (hereinafter “FATC”) in a tibial alignment following the methods of Woo et al. with modifications to accommodate ultrasound imaging. The femur and tibia were aligned along the tensile axis with 0° of knee flexion, and the sample was submerged in degassed physiologic saline. In addition to preventing tissue dehydration, the saline provided a medium for ultrasound propagation. A preload of 2 N was applied for one minute, and the joint was preconditioned by cyclic sawtooth loading from 0-0.75 mm for 10 cycles at 20 mm/min followed by a rest of 1 min. Three load regimens were applied to each sample (n=3). First, the joint was cyclically loaded from 0-2 mm at 20 mm/min, with 0 mm being the displacement during the preload. Following a 30 minute rest, the joint was cyclically reloaded from 0-3 mm, with additional displacement applied during this testing regimen to ensure a detectable amount of deformation occurred across the insertions. Finally, after an additional 30 minute rest, the joint was loaded to failure at 10 mm/min.

### Materials and Methods for Ultrasound Data Collection and Processing

While the joints were loaded in tension, an ultrasound scanner (Terason 2000; Teratech, Inc., Rockville, Md.) acquired Radio Frequency (RF) data at 5 MHz using a linear array. The ultrasound transducer was mounted inside the saline tank and positioned to image the ACL and insertions. Sequences of ultrasound RF data were acquired continuously during the applied loading repeatedly for periods of 3 seconds at 54 frames/s (128 RF lines, sampling frequency: 10 MHz). The axial displacement between a reference and successive frames was estimated offline and imaged using cross-correlation and recorrelation techniques with a window size of 3 mm and a window overlap of 80%.

Axial displacements, or displacements occurring in the direction orthogonal to the face of the transducer and parallel to the direction of ultrasound propagation, were estimated for each RF frame with respect to a reference frame using a 1D cross-correlation algorithm. In this algorithm, time-shifts between two backscattered signals are determined by the cross-correlation of small sliding windows over the entire 2D ultrasound image. At high decorrelation noise, recorrelation techniques were employed. Finally, the strain distribution was computed by differentiating the displacement map along the axial direction. For the numerical differentiation, a least-squares regression method was used. Displacement and strain were estimated relative to a reference frame, which was captured at the beginning of the application of tensile load, in order to obtain a temporal profile and map of the cumulative deformation at the ligament and the insertion.

### Results Obtained

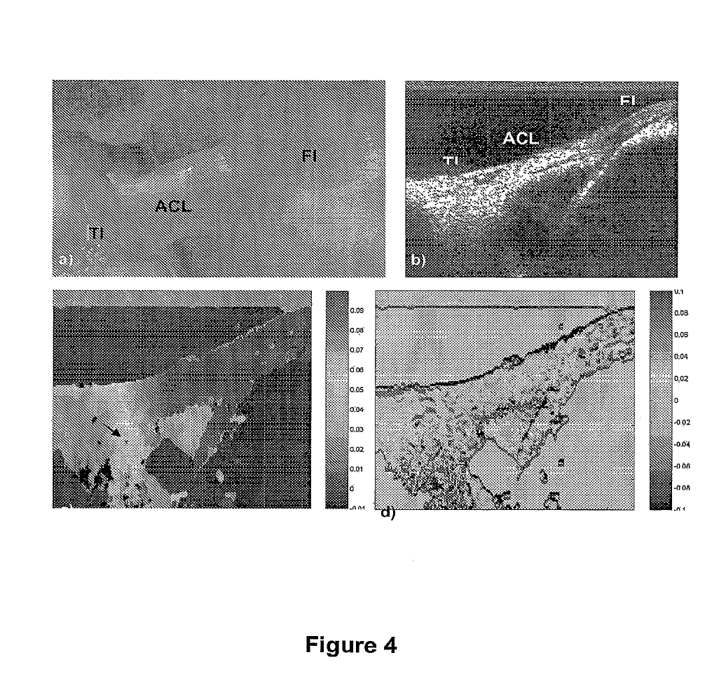

In all specimens tested, the ACL and the interface between the ACL and the femoral or tibial bone (FIG. 4A) were readily identifiable on the ultrasound images (FIG. 4B), as well as in all subsequent displacement maps and strain elastograms. Each tissue type exhibited a distinct ultrasound signature based on the structure and density of the tissue, which varies from soft to hard tissues and results in distinct speckle patterns distinguishing ligament, cartilage, and bone. As a highly dense tissue, bone is strongly echogenic resulting in bright ultrasound reflection and poor propagation of ultrasound waves through bone. Ligament and bone are less dense and therefore less echogenic, enabling the structure of these tissues to be discernable on the B-mode images. Within the ACL, a narrow band of high strain in the middle and along the length of ACL was noted that also corresponded to a highly echogenic area on the B-scan images. This may reflect the parallel bundle-organization of the ACL. Distinctions between soft and hard tissue signatures were used to identify the ACL insertions into bone.

After establishing the ability to image the ACL and insertions with the experimental setup, mechanical testing was performed in order to obtain the mechanical properties of the FATC using traditional mechanical testing means, as well as to determine the localized mechanical behavior of the ACL and tibial insertion using ultrasound elastography. For neonatal bovine FATCs tested in a tibial orientation with 0° of knee flexion (FIGS. 3A and B), the average stiffness was 59±15 N/mm and the average modulus was 100±30 MPa. Elastographic analysis yielded maps of cumulative deformation and strain in the ACL and tibial insertion during tensile loading. FIG. 4C shows the distribution of deformation throughout the FATC, with magnitudes of deformation represented according to a colormap, with small deformations blue and large deformations red. The magnitude of displacement was found to be the highest (red in FIG. 4C) within the ACL proper and decreased in value in a gradual transition (orange, yellow, and green) from ligament (red) to bone (blue). In addition, elastographic analysis revealed through strain maps that the strain profile at the tibial insertion was highly complex as the FATC was loaded in tension (FIG. 4D). Both compressive and tensile strains were visualized at the tibial insertion site, indicated by the green-blue and yellow-red regions, respectively, on the elastogram in FIG. 4D. In addition, compressive strains (blue) were found in the ACL itself, most likely because in this experiment, the ultrasound transducer was aligned with respect to the insertion (region of interest) during loading. A narrow region of blue appears along the entire upper tissue-saline interface in the elastogram in FIG. 4D, which is an artifact of the high acoustical impedance difference between the FATC and saline and should not be confused with compressive strain.

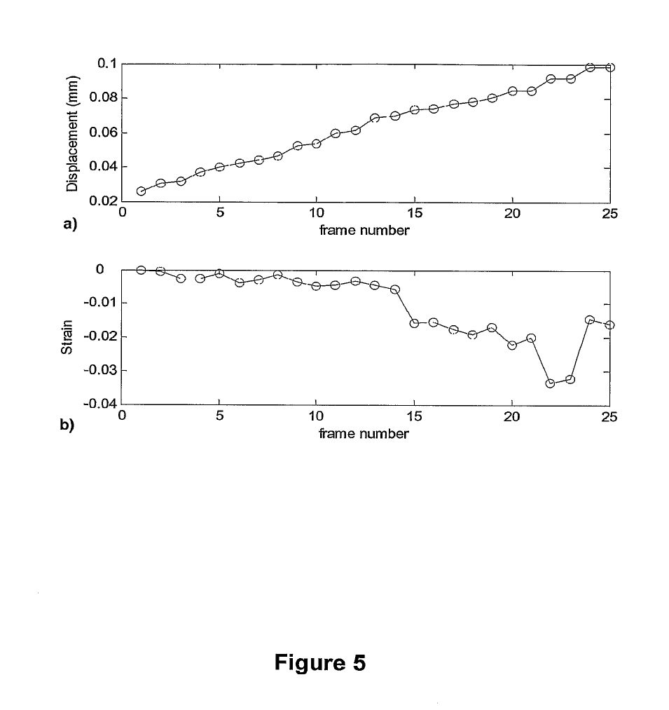

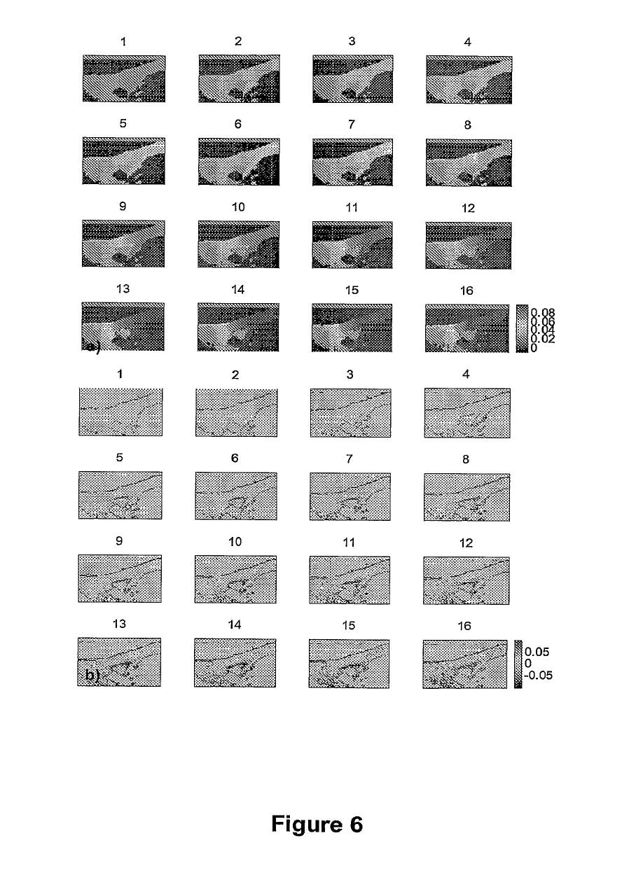

Point-wise temporal displacement and strain analyses at the tibial ACL insertion demonstrate the deformation at the insertion over time with the development of both tensile and compressive strain. As shown in FIGS. 5A and 5B, which depicts the displacement and strain response, respectively, at a single point at the tibial insertion undergoing compressive strain (FIG. 4D, arrow), a constant rate of deformation was observed, with a corresponding non-constant evolution of compressive strain. The accumulation of deformation and strain throughout the entire FATC can be seen in FIG. 6, which shows deformation (FIG. 6A) and strain (FIG. 6B) for a sequence of 16 frames with respect to a single reference frame. While the magnitude of deformation remains relatively constant in the bone (blue) over time from frames 1-16, the ACL proper undergoes high deformation, as shown be the change in color of the ACL from blue to green, yellow, orange, and finally red (FIG. 6A). Additionally, FIG. 6B reveals the evolution of strain over time, indicating the development of complex strain with both compressive (blue) and tensile (red) components at the interface between the ACL and bone.

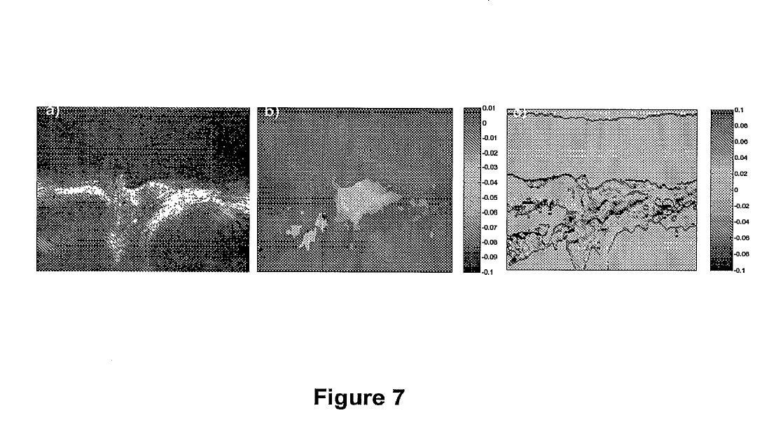

In the elastographic analysis method used in this study, displacement and strains are measured along the axis parallel to the direction of ultrasound beam propagation. This may introduce artifacts corresponding to the orientation of the transducer with respect to the FATC. To ensure that results were not dependent on the orientation of the transducer, an additional trial was performed with the transducer rotated such that the face of the transducer was aligned along the principal axis of the ACL (FIG. 7A). It is important to note that in this sample, a gradual decrease in the magnitude of deformation was again observed across the insertion, with high deformation (green) in the ACL, a low degree of deformation in bone (dark red), and a transition from green to yellow, orange and red at the interface between the ACL and bone (FIG. 7B). Additionally, compressive strain (blue regions, FIG. 7C) is again observed at the interface while the joint is loaded in tension. Tensile strains (red regions) are found at the same interface. These results are consistent with those presented in FIG. 4 and combined collectively demonstrate the complexity of the strain profile at the tibial insertion.

These results demonstrate that displacement is non-uniformly distributed throughout the FATC and that strain in the ACL insertions is complex. Displacement images reveal that deformation is higher in the ACL midsubstance compared to the tibial insertion, and that a gradual transition exists in the degree of deformation from the ligament proper, through the tibial insertion, into bone. This distribution indicates a tissue type-dependent increase in stiffness progressing from ligament to interface and then to bone. Strain elastograms revealed that the strain distribution at the tibial insertion is highly complex, with both tensile and compressive strain components localized at the tibial insertion site. The complexity of strain distribution within the insertions may be due to the transfer of tensile strain from the ligament to bone through the interfacial fibrocartilage tissue found at direct ligament and tendon insertions.

The results obtained by methods of the present invention and described herein constitute the first experimental determination of the complex strain distribution at ACL insertion sites and allow for the following observations: First, the presence of a fibrocartilaginous transitional tissue between ligament and bone demonstrates that a compressive strain component exists in that region during physiological loading. Second, collagen fibers extending from ligament into bone at the insertions, when loaded in tension, transmit shear and compressive stresses through the fibrocartilage zones of the insertions.

These results allow for the determination of mechanical properties of the ACL and ACL-bone interface and represent a milestone in the understanding of the localized functionality of orthopaedic tissues, specifically at soft to hard tissue interfaces. The results obtained from the above-described methods also inform the engineering of interfacial scaffolds based on data derived from the analysis of the mechanical properties of healthy ACL insertions. In addition these methods allow for quantitative and noninvasive evaluation of the success of efforts to improve graft to bone healing.

These results demonstrate that these methods of ultrasound elastography provide valuable information on the mechanical behavior of the ACL insertions upon physiological loading. This information allows for a better understanding of the structure-function relationship inherent at the ACL-bone interface, as well as tendon graft healing and re-establishment of a functional tendon-bone interface. In addition, mechanical parameters, such as Young's modulus, shear modulus and Poisson's ratio can be estimated and imaged based on the elastographic measurements.

### Example 2

Techniques applied in Example 1 to anterior cruciate ligaments in vitro, are applied in situ to characterize ACL ligaments. Highly detailed data characterizing strain responses of ACL ligaments are obtained.

### Example 3

Techniques applied in Example 1 to anterior cruciate ligaments are applied to other structures of the tibiofemoral joint, in vitro and in situ, including the posterior cruciate ligament, cartilage, and medial and lateral menisci. Highly detailed data characterizing strain responses of these structures are obtained.

### Example 4

Techniques applied in Example 1 are applied to other joints of the upper and lower extremities including the foot, ankle, hip, temporomandibular joint (TMJ), shoulder, elbow, hand and wrist, in vitro and in situ. Highly detailed data characterizing strain responses of structures associated with these joints are obtained.

### Example 5

Techniques applied in Example 1 are applied to tendons, such as the achilles tendon and flexor and extensor tendons of mammalian extremities, in vitro and in situ. Highly detailed data characterizing strain responses of tendons are obtained.

### Example 6

### Materials and Methods

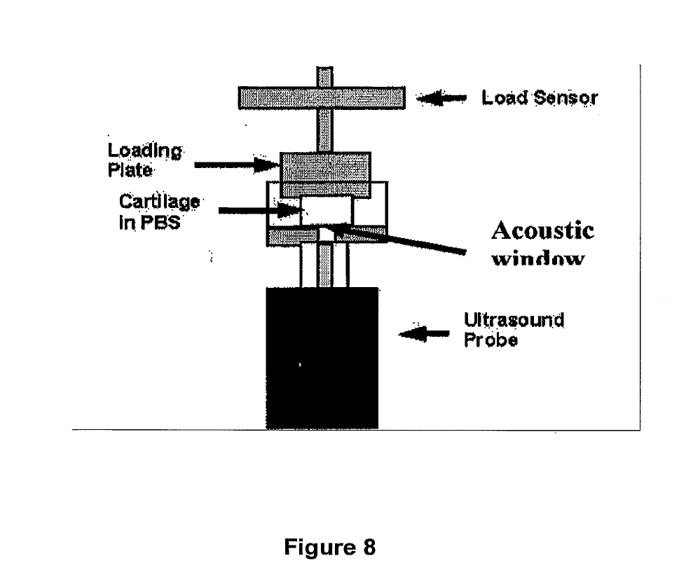

Full-thickness, 1-cm diameter cylindrical samples of articular cartilage (n=3) were obtained from bovine, femoral condyles with an average thickness of 4.71±0.47 mm and femoral head with an average thickness of 3.39±0.86 mm in immature, healthy bovine. The samples were immersed in PBS within a custom-made loading device, illustrated in FIG. 8, mounted onto an Instron 5848 MicroTester (Norwood, Mass.) equipped with a 100 N load cell (accuracy +/−0.5N, with a 1-micron displacement resolution of the actuator).

The specimens were oriented such that the deep portion of the cartilage contacted an aluminum loading plate and the articular surface rested upon another rigid, impermeable surface containing a 3 mm opening to serve as the acoustic window for the high-resolution ultrasound transducer (f/2, 8 mm focus, 55 MHz, 46 Hz frame rate, Vevo 770, Visualsonics, Toronto, Canada). The ultrasound probe was separated by 3-mm from the surface of the articular cartilage.

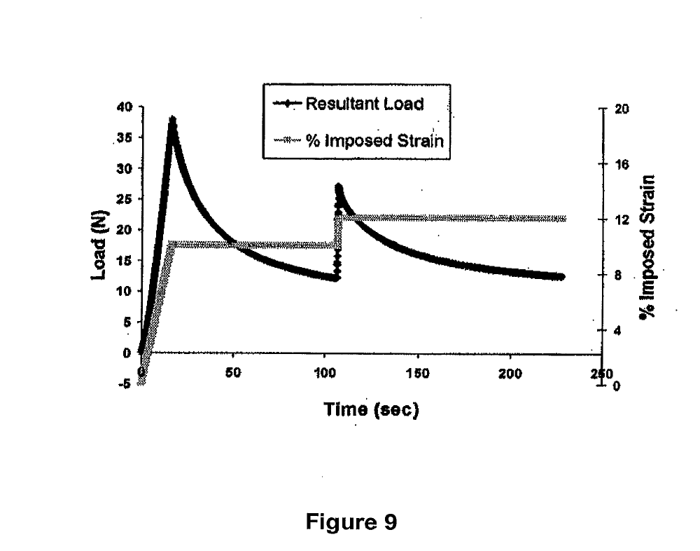

A tare strain of 0.1% based on the measurement of the undeformed cartilage plugs was sustained for 30 seconds, followed by a ramp to strains ranging from 0.5 to 4.0% strain at 0.1 mm/sec for two femoral condyle samples and one femoral head sample, the results of which are graphically described in FIG. 9. For these samples, B-mode ultrasound scans were acquired immediately after the tare strain and immediately after ramped compression of the cartilage. Subsequently, a pre-strain of 10% based on the measurement of the undeformed cartilage plugs was applied for 30 seconds, followed by a ramp to strains ranging from 2% at 0.1 mm/sec for two femoral head samples. Radiofrequency (RF) ultrasound signals of these samples were acquired once equilibrium was attained. Displacement images and elastograms were generated using ID crosscorrelation techniques and gradient operators on the RF signals (window size 0.3 mm, 85% overlap), respectively. Median filtering of the displacement data was also implemented. This arrangement simulates a device in which an ultrasound transducer is incorporated into an arthroscopic indentation device.

### Results Obtained

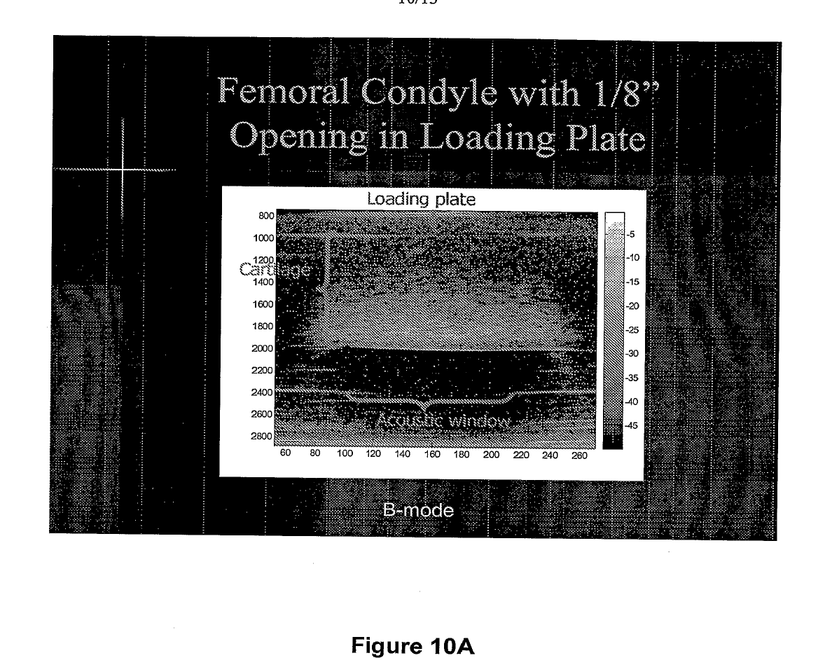

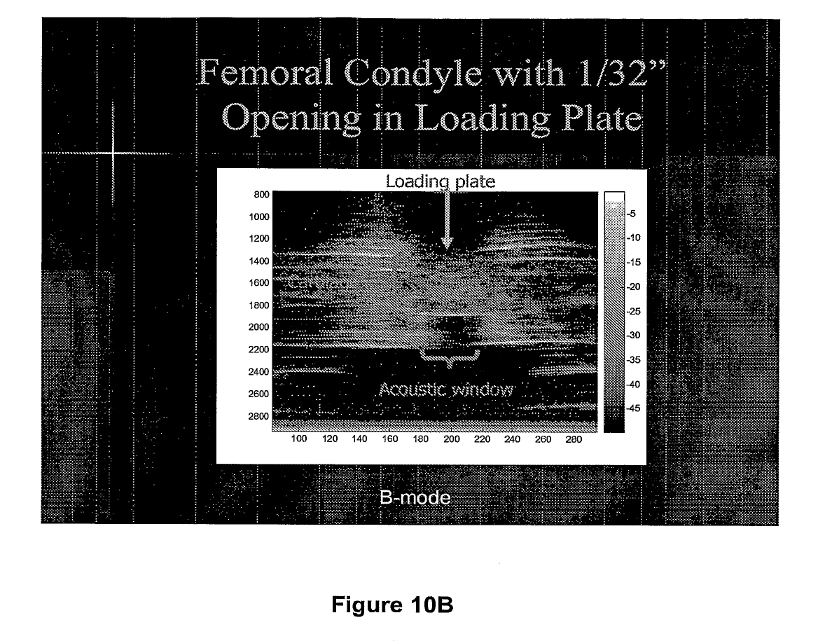

As shown in FIG. 9, ramp compressions with high resolution displacement were applied and rapid stress relaxation of the articular cartilage samples recorded. As shown in FIGS. 10A and 10B, high quality RF signals and B-mode images were obtained of the cartilage plugs using the high resolution ultrasound with a 1/16″ (FIG. 10A) and 1/32″ (FIG. 10B) diameter opening in the loading plate. The full thickness of the cartilage sample can be observed and the articular layer can be discerned from zone 2. Abundant speckle is also visible within the cartilage, thus facilitating displacement and strain imaging.

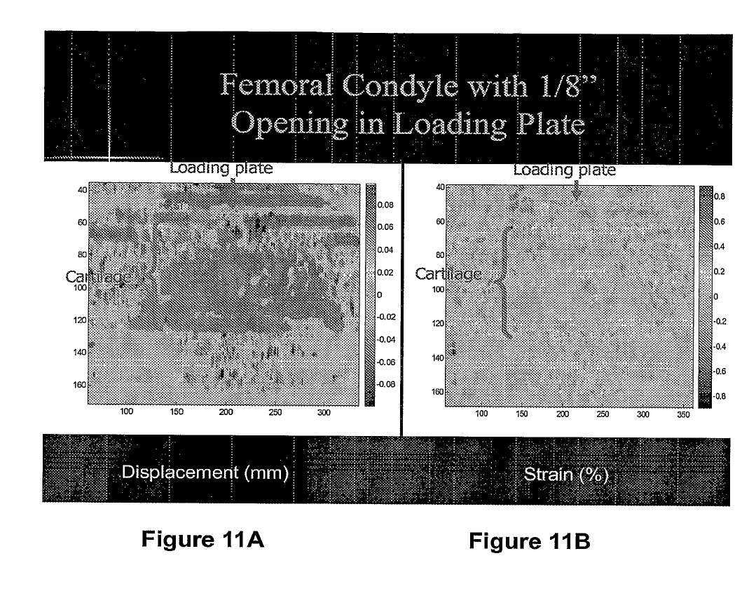

As shown in FIG. 11A, displacement images for all the samples imaged using the ⅛″ opening in the loading plate revealed that while displacement of the region of interest did occur, a gradient of the displacements did not result. Rather, it appears that the entire region of the sample underwent rigid motion, displacing upwards through the opening as evidenced by circular indentations which were observed on the cartilage surfaces following the loading protocol. As shown in FIG. 11B, the corresponding elastogram therefore displayed zero strain within most of the sample. Only the articular surface and the interface exhibited local strain. Similar maps were obtained regardless of the extent of post-compression strain.

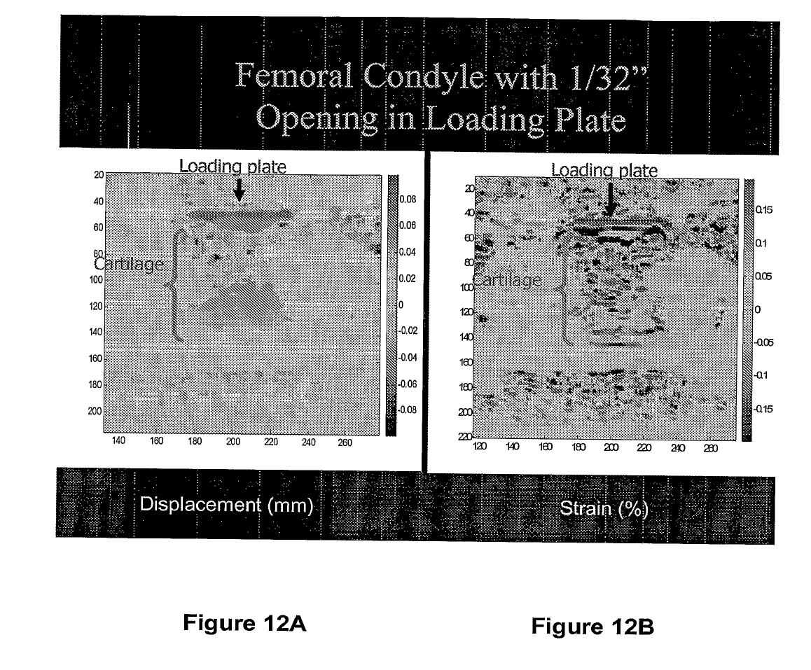

Consistent with FIG. 12A, the majority of displacement images for all the samples imaged using the 1/32″ opening in the loading plate revealed that while displacement of the region of interest did occur, local displacements did not result. As shown in FIG. 12B, the corresponding elastogram displayed about 0.5% strain within a portion of zone 2, while the articular surface and the interface exhibited strains on the order of 0.2%.

Whether the RF signals were captured immediately after the compressions or once equilibrium was achieved did not significantly impact the appearance of the cartilage on the elastograms. The elastograms were also similar between femoral condyle specimens and femoral head samples.

This Example confirms the usefulness of high resolution ultrasound elastographic imaging of articular cartilage, for example, for the early diagnosis and monitoring of treatment for articular cartilage pathologies, such as osteoarthritis.

### Example 7

Techniques applied in Example 6 to articular cartilage in vitro, are applied in situ. Highly detailed data images characterizing local strains to articular cartilage are obtained.

### Example 8

Techniques applied in Example 6 are applied to articular cartilage of the joints of the upper and lower extremities including the ankle, hip, shoulder, elbow and wrist, in vitro and in situ. Highly detailed data images characterizing local strains of articular cartilage associated with these joints are obtained.

It will be understood that the foregoing description is illustrative of the principles of the invention, and that various modifications can be made by those skilled in the art without departing from the scope and spirit of the invention.

What is claimed is:

1. A method for obtaining information about the mechanical behaviour of structures associated with mammalian joints comprising:
(a) creating deformation in a joint structure of interest;
(b) using an ultrasound scanner in a linear array to acquire sequences of ultrasound data of the joint structure; and
(c) estimating a displacement component or displacement distribution between a reference frame of backscattered signals and a successive frame of backscattered signals, wherein said displacement component is estimated with a matching algorithm.

2. The method of claim 1, further comprising repeating step (c) until sufficient data is obtained to estimate strain distribution in the joint structure of interest.

3. The method of claim 2, wherein the joint structure of interest is selected from the group consisting of: ligaments and articular cartilage and any combination thereof.

4. The method of claim 3, wherein the matching algorithm includes determining time-shifts between two RF signals by cross-correlating sliding windows over a 2D or 3D ultrasound image to provide an estimation of axial, lateral or elevational displacement components.

5. The method of claim 4, further comprising computing a strain or strain rate distribution.

6. The method of claim 5, wherein computing a strain or strain rate distribution comprises differentiating a displacement map along one of the principle directions.

7. The method of claim 6 wherein the said differentiating comprises numerical differentiation, wherein the numerical differentiation includes least-square regression.

8. The method of claim 1, wherein said estimation of the displacement component further includes a recorrelation algorithm.

9. The method of claim 1 or 8, further comprising the use of a window size between about 1 and about 5 mm and a window overlap between about 50 and about 99%.

10. The method of claim 1, wherein the joint structure of interest is associated with the tibiofemoral joint.

11. The method of claim 10, wherein the ligament is an anterior cruciate ligament.

12. The method of claim 10, wherein the ligament is a posterior cruciate ligament.

13. The method of claim 1, wherein the information is obtained in vitro.

14. The method of claim 1, wherein the information is obtained in vivo.

15. The method of claim 1, wherein the information is obtained in situ noninvasively.

16. The method of claim 1, wherein the information is obtained in situ during a minimally invasive procedure such as an arthroscopy.

17. The method of claim 1, wherein the information is obtained in situ during an invasive procedure such as hip surgery.

18. The method of claim 1, wherein the joint structure of interest is associated with a joint selected from the group consisting of: foot, ankle, knee, hip, hand, wrist, elbow, shoulder and temporomandibular joint (TMJ).

19. The method of claim 1, wherein the mammal is selected from the group consisting of: human, equine, canine and feline.

20. The method of claim 1 or 2, wherein said information is used to inform the design of tissue grafts.

21. A tissue graft produced using the information obtained by the method of claim 1 or 2.

22. A method for obtaining information about the mechanical behaviour of mammalian tendons comprising:
(a) creating deformation in a tendon of interest;
(b) using an ultrasound scanner with a piezoelectric element or an array of piezoelectric elements to acquire sequences of ultrasound data of the tendon; and
(c) estimating the displacement between a reference frame of the ultrasound data and successive frames of the ultrasound data, wherein said displacement or displacement distribution is estimated with a cross-correlation algorithm.

23. The method of claim 22, further comprising repeating step (c) until sufficient data is obtained to estimate strain distribution in the tendon of interest.

24. The method of claim 22, wherein the cross-correlation algorithm includes determining time-shifts between two backscattered RF signals by cross-correlating sliding windows over a 2D or 3D ultrasound image to provide an estimation of axial, lateral or elevational displacement components ultrasound image.

25. The method of claim 24, wherein the cross-correlation algorithm includes determining time-shifts between two backscattered RF signals by cross-correlating sliding windows over a 2D or 3D ultrasound image to provide an estimation of axial, lateral or elevational displacement components ultrasound image.

26. The method of claim 24, wherein the cross-correlation algorithm includes determining time-shifts between two backscattered RF signals by cross-correlating sliding windows over a 3D ultrasound image to provide an estimation of axial, lateral or elevational displacement components ultrasound image.

27. The method of claim 23, further comprising computing a strain or strain rate distribution.

28. The method of claim 27, wherein computing a strain or strain rate distribution comprises differentiating a displacement map along the axial direction.

29. The method of claim 28 wherein said differentiating comprises numerical differentiation and wherein the numerical differentiation includes least-square regression.

30. The method of claim 22, wherein said estimation of axial displacement further includes a recorrelation algorithm.

31. The method of claim 23 or 28, further comprising the use of a window size between about 1 and 5 mm and a window overlap between about 50 and 99%.

32. The method of claim 1 or 22, wherein said deformation is selected from the group consisting of tension, compression and relaxation, or any combination thereof.

33. The method of claim 1 or 22, wherein said deformation is generated by the ultrasound probe itself.

34. The method of claim 1 or 22, wherein said deformation is generated by the scanned subject itself.

35. The method of claim 22, wherein said piezoelectric element is embedded or is part of a surgical tool.
